(19) United States
(12) Patent Application Publication
(10) Pub. No.: US 2005/0121038 A1
(43) Pub. Date: Jun. 9, 2005

(54) Method and apparatus for pharyngeal augmentation of ventilation

(75) Inventor: Kent L. Christopher, Denver, CO (US)

Correspondence Address: DORR CARSON SLOAN & BIRNEY, PC, 3010 EAST 6TH AVENUE, DENVER, CO, 80206, US

(73) Assignee: CS Medical, Inc.

(21) Appl. No.: 11/012,841

(22) Filed: Dec. 15, 2004

Related U.S. Application Data
(63) Continuation of application No. 09/818,228, filed on Mar. 27, 2001.
(60) Provisional application No. 60/200,030, filed on Apr. 26, 2000.

(51) Int. Cl. A62B 7/00; A61M 16/00

(52) U.S. Cl. 128/207.18; 128/207.14

(57) ABSTRACT

A nasopharyngeal catheter provides direct pharyngeal delivery of high flows of humidified air, oxygen, helium, or other gases to supplement ventilation of a spontaneously breathing patient. For example, flow rates in the range of approximately 4 to 40 liters per minute can be employed. The flow passes through a heater that maintains a desired temperature, and a humidifier that maintains a desired relative humidity. The present invention includes a nasal catheter that can be cut to a desired length and removably attached to a horizontal delivery tube. Gas can be supplied through oxygen connections at either end of the horizontal delivery tube.

### RELATED APPLICATION

The present application is based on, and claims priority to the Applicant's U.S. Provisional Patent Application Ser. No. 60/200,030, entitled “Method and Apparatus for Pharyngeal Augmentation of Ventilation,” filed on Apr. 26, 2000.

### BACKGROUND OF THE INVENTION

1. Field of the Invention

The present invention relates generally to the field of systems for augmenting patient ventilation. More specifically, the present invention discloses a nasopharyngeal catheter used for providing a supplemental flow of air/oxygen to a patient.

2. Statement of the Problem

A variety of conventional therapies are currently used for treatment of sleep apnea. A mechanical ventilation system can be used to supply nasal continuous positive airway pressure (CPAP) or bilevel positive airway pressure (BiPAP) through a mask strapped to the patient's face. Both CPAP and BiPAP ventilation seek to achieve elevated backpressures to relieve airway obstruction. These therapies can be effective in a clinical setting (e.g., in a sleep laboratory), but tend to suffer from poor compliance in the home due primarily to patient discomfort and the obtrusiveness of the required equipment. In particular, patients often experience discomfort due to the drying effect on the airway, the sensation of pressure, and other adverse effects from the mask, such as cellulitis, nasal congestion, dermatitis, nose bleeds, and claustrophobia. The obtrusiveness of the face mask, large-bore tubing, ventilator noise, restricted sexual activity, and restricted mobility associated with these therapies impacts both the patient and the patient's significant other.

Surgical procedures are sometimes used to treat sleep apnea. Nasal surgery tends to have minimal long-term benefits. A uvulopalatalpharyngoplasty (UPPP) can be performed if the obstruction is thought to be at the level of the soft palate (i.e., between the nasopharynx and the oropharynx) However, this surgical procedure is relatively uncomfortable and has a relatively low success rate, typically not greater than 50 percent.

Supplemental air/oxygen can be delivered via a nasal cannula.

This more comfortable and less obstructive than surgery or CPAP/BiPAP ventilation. But, it is not effective in treating obstructive sleep apnea, even with high flows, since the flow merely exits through the patient's nostrils if an airway obstruction exists.

Tracheotomy is generally successful in treating sleep apnea, but is extremely poorly tolerated due to daytime inconvenience, interference with normal speech, patient discomfort, and poor social acceptance.

Finally, ventilation can be augmented via a transtracheal catheter. This approach allows either low or high flows of humidified gas to be delivered directly into the patient's trachea. It is very effective, relatively comfortable for the patient, and only minimally intrusive. In addition to relieving the obstruction in sleep apnea, transtracheal augmentation of ventilation with high flows of heated and humidified air has been shown to offer the advantages of reduced physiologic dead space, reduced inspired minute ventilation, decreased work of breathing, improved daytime activity and exercise capacity, and improved sleep for the patient. However, it does require surgery for creation of a tracheal stoma, and involves inconvenience for maintenance and care, including keeping the stoma open both day and night.

Similarly, conventional therapies for treatment of respiratory failure or insufficiency have many of the same shortcomings. Nasal CPAP and BiPAP ventilation have the same issues and concerns as with sleep apnea. Rather than relieving the obstruction, these therapies are intended to “rest” respiratory muscles and reduce the work of breathing. Little data are available to show any resulting long-term benefits, but the patient may have a reduced PCO₂. As previously discussed, CPAP and BiPAP ventilation often causes patient discomfort due to the drying effect that flows of unhumidified air/oxygen can have on nasal and pulmonary secretions. The patient may also feel claustrophobic and may “fight” the efforts of the device to force air/oxygen into the nose. The previously discussed shortcomings associated with a tracheotomy with conventional mechanical ventilation or transtracheal augmentation of ventilation also apply in treatment of chronic or acute respiratory failure or insufficiency.

Previous Nasopharyngeal Catheters. Nasopharyngeal catheters were formerly used to deliver low flow rates of oxygen to hospitalized patients. A length of flexible tubing was inserted into the patients' nostril and its distal tip was advanced through the nasal cavity into the nasopharynx until it could be viewed past the soft palate by looking into the patient's mouth. The catheter was then withdrawn until it disappeared behind the soft palate. The tubing was held in place by tape applied to the bridge of the patient's nose. The state of technology at that time only allowed for delivery of poorly humidified gas. As a result, mucus would tend to obstruct the catheter. The catheter would have to be removed, cleaned, and reinserted every eight hours, which often resulted in poor patient tolerance. In addition, the catheter could be easily dislodged out of the patient's nose or inadvertently advanced into the patient's esophagus, potentially causing serious complications such as gastric distention, perforation, and aspiration. The catheter could also be inadvertently placed into the trachea or lungs. Due to these shortcomings, this technology has not been used for approximately 30 to 40 years.

3. Solution to the Problem

The present invention provides a method and apparatus for direct pharyngeal delivery of high flows of humidified air, oxygen, or other gases to supplement ventilation of a spontaneously breathing patient. For example, the present invention can be used for the purpose of treating patients with respiratory failure or insufficiency, or sleep apnea syndrome. In a home setting, the present invention can be employed for nocturnal augmentation of patients with sleep apnea syndrome (obstructive, central, or mixed), or chronic respiratory failure or insufficiency resulting from emphysema (COPD), other obstructive lung diseases, interstitial lung diseases, pleural diseases, neuromuscular diseases, and other restrictive disorders. In a hospital setting, the present invention can be used to treat patients with acute respiratory failure/insufficiency or acute respiratory failure/insufficiency superimposed upon chronic respiratory failure/insufficiency. The present system can be used intermittently or throughout the day and night to augment ventilation and avoid the need for endotracheal intubation and conventional mechanical ventilation.

The present invention offers a number of advantages over the prior art in treatment of sleep apnea and respiratory failure/insufficiency. No surgical procedure is required. The device is more comfortable and less obtrusive for the patient to wear. The catheter effectively bypasses any obstructions in the patient's nasal cavity and nasopharynx. The high flow of gas can also helps to relieve any obstruction between the nasopharynx and trachea (e.g., obstruction by the tongue). The flow of air/oxygen is thoroughly humidified, which reduces accumulation of mucus and drying of the patient's airway. There are no constraints on the patient during periods when the patient is not receiving therapy. In addition, the present system can be used to deliver a variety of gases including air (for sleep apnea and neuromuscular disorders), air and oxygen (for hypoxemia), and helium and oxygen (for enhanced gas transport and other physiologic benefits, such as reduced work of breathing).

### SUMMARY OF THE INVENTION

This invention provides a nasopharyngeal catheter for direct pharyngeal delivery of high flows of humidified air, oxygen, helium, or other gases to supplement ventilation of a spontaneously breathing patient. For example, flow rates in the range of approximately 4 to 40 liters per minute can be employed. The flow passes through a heater that maintains a desired temperature, and a humidifier that maintains a desired relative humidity. The present invention includes a nasal catheter that can be cut to a desired length and removably attached to a horizontal delivery tube. Gas can be supplied through oxygen connections at either end of the horizontal delivery tube.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a block diagram of the present system including a vertical cross-sectional view of a patient's upper airway with the nasopharyngeal catheter in place.

FIG. 2 is a front elevational view of the patient's face and the nasopharyngeal catheter.

FIG. 3 is a top plan view of the nasopharyngeal catheter.

FIG. 4 is a top plan view of the nasopharyngeal catheter with the nasal catheter 22 detached from the horizontal delivery tube 20.

FIG. 5 is a perspective view of the nasopharyngeal catheter.

FIG. 6 is a side elevational view of the oxygen connecting tube 30.

FIG. 7 is a side elevational view of the cleaning rod 40 and connecting hose 30.

### DETAILED DESCRIPTION OF THE INVENTION

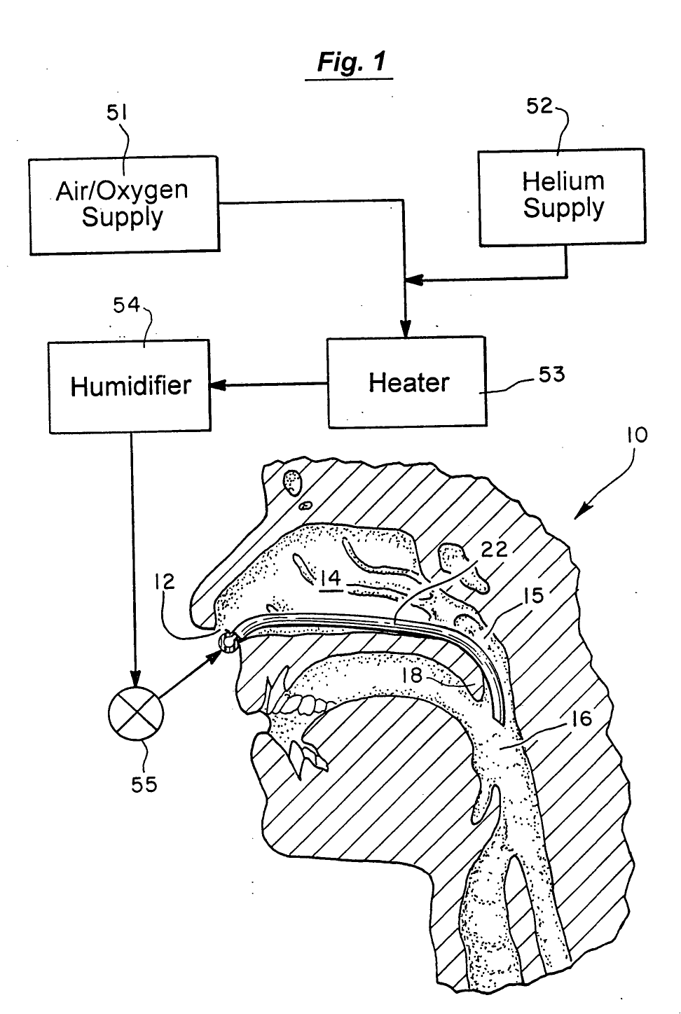

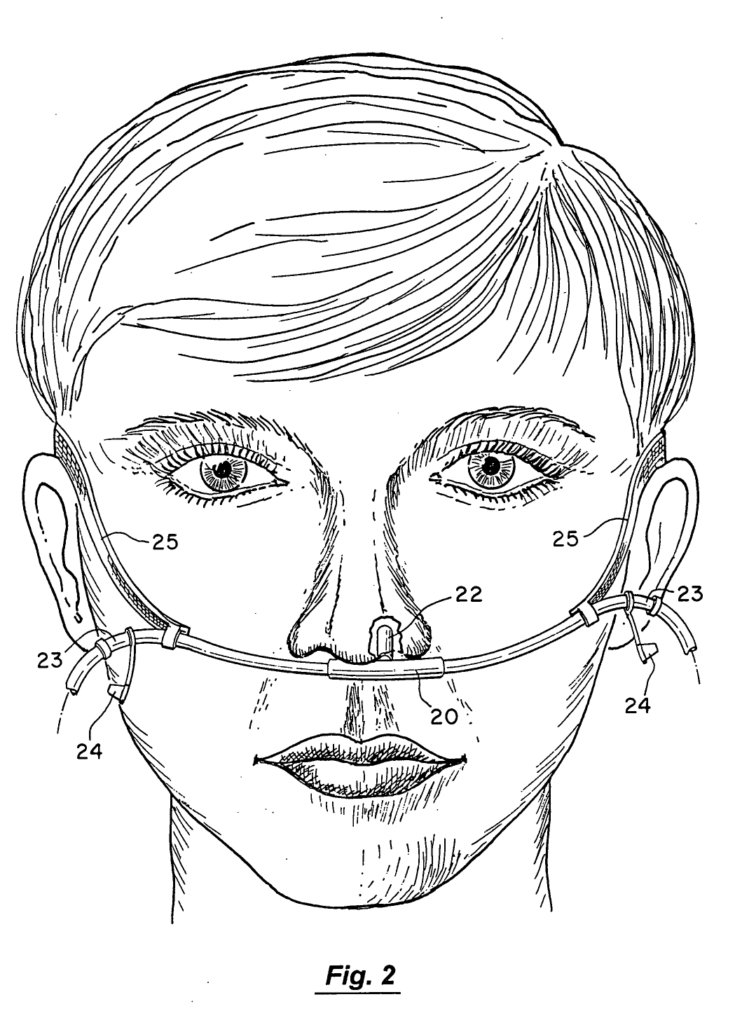

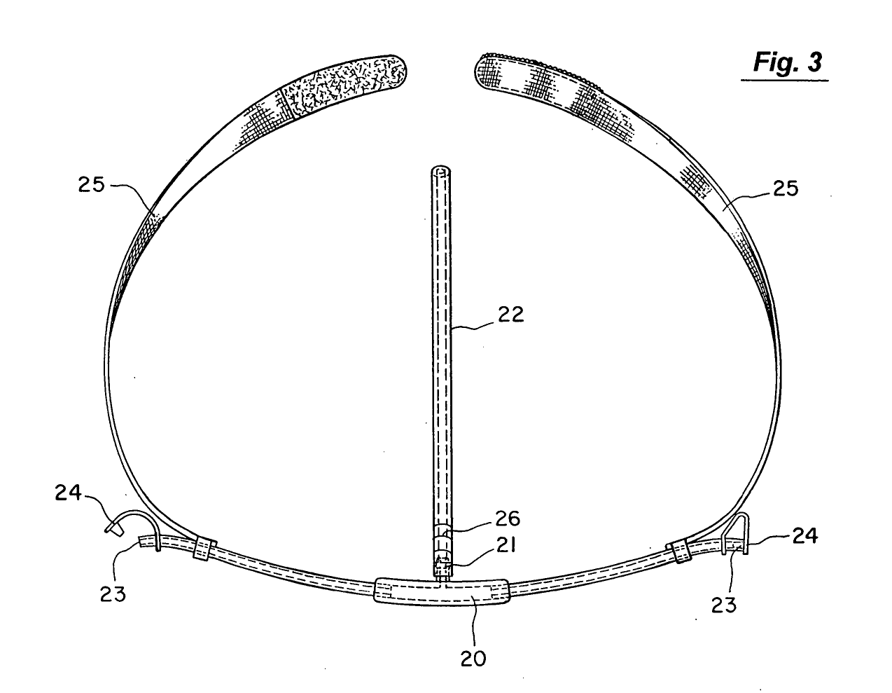

Turning to FIG. 1, a block diagram is provided of the present system including a vertical cross-sectional view of a patient's upper airway 10 with the nasopharyngeal catheter in place. FIG. 2 is a corresponding front elevational view of the patient's face and the nasopharyngeal catheter. As depicted in the top plan view of the nasopharyngeal catheter shown in FIG. 3, the present device includes a horizontal delivery tube 20 that is preferably made of soft, clear PVC or silicone tubing (approximately 75-85 A durometer) having an inside diameter of approximately 4 to 5 mm, and an outside diameter of approximately 5 to 6 mm.

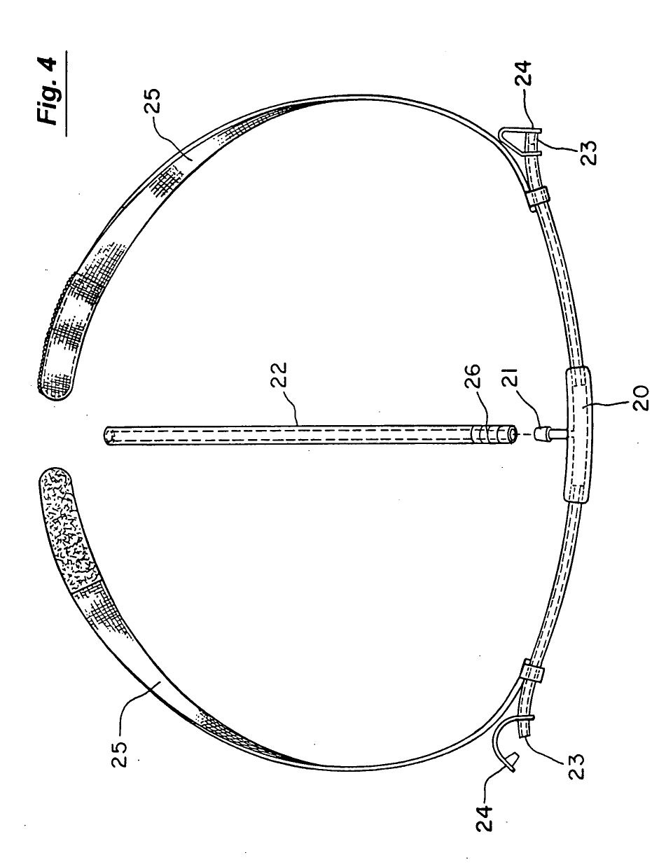

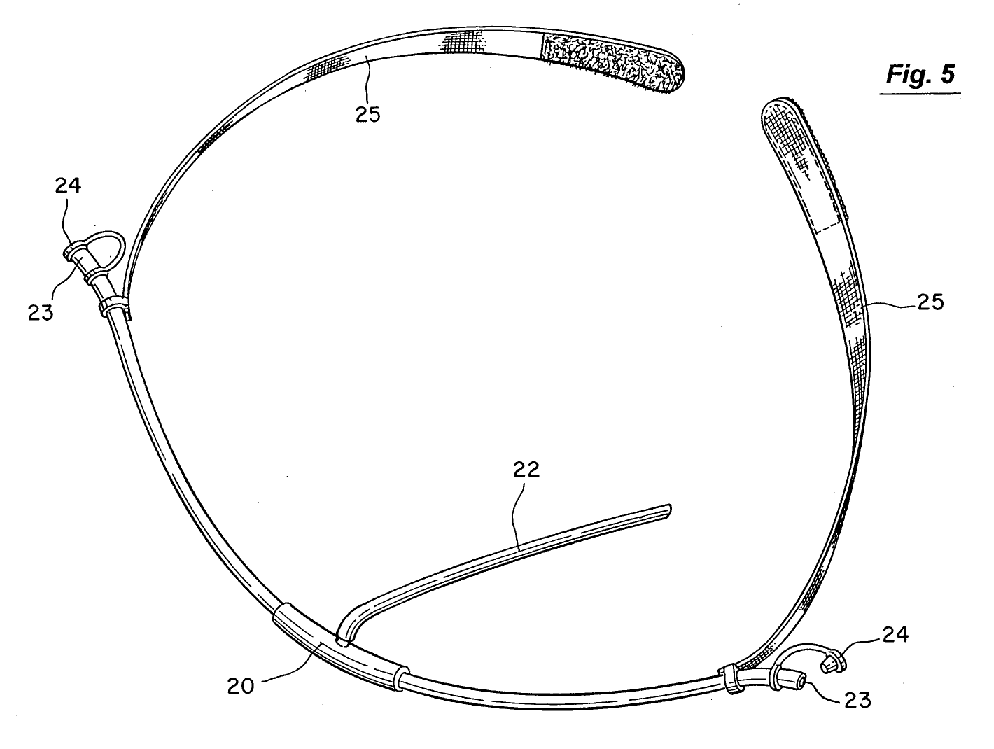

A nasal catheter 22 can be removably attached to a barbed connector 21 on the delivery tube 20, as illustrated in FIG. 4. The nasal catheter 22 can be bent or contoured, as shown in the perspective view provided in FIG. 5, to approximate the contour of the nasal cavity 14 and nasopharynx 15. The nasal catheter 22 is preferably made of soft, clear PVC or silicone tubing (approximately 92 A durometer) having a length of approximately 20 cm, an inside diameter of approximately 3 mm (9 French), and an outside diameter of approximately 4 mm (12 French). A hydrophilic coating helps to prevent adherence of mucus to the nasal catheter 22. A viscous lidocaine coating can also be applied to the exterior of the nasal catheter 22 to reduce patient discomfort. The distal tip of the nasal catheter 22 is rounded with a smooth inside and outside diameter to minimize discomfort during insertion of the nasal catheter 22. A series of markings 26 are placed on the proximal portion of the nasal catheter in 5 mm increments as shown in FIG. 4.

The distal tip of the nasal catheter 22 is inserted through the patient's nostril 12 and advanced into the nasal cavity 14 and nasopharynx 15 until it is visible through the patient's mouth below the uvula 18 in the upper portion of the oropharynx 16, as shown in FIG. 1. If necessary, the healthcare provider can adjust the position of the catheter tip relative to the patient's uvula by observation through the patient's mouth. The healthcare provider then notes the appropriate length for the nasal catheter 22 by observing the position of the markings 26 relative to the patient's nostril 12. The nasal catheter 22 is then withdrawn to a predetermined degree (e.g., slightly) and its proximal end is cut to the desired length relative to the markings 26. This feature allows the nasal catheter 22 to accommodate a wide variety of patient dimensions. The proximal end of the nasal catheter is then attached to the barbed connector 21 on the delivery tube and reinserted. After the nasal catheter 22 has been reinserted, its distal end typically extends into either the distal nasopharynx or oropharynx.

Alternatively, a fixed-length nasal catheter 22 can be permanently attached to the delivery tube 20. The healthcare provider would then select a device having a nasal catheter 22 of appropriate length for each patient. Optionally, a radio-opaque stripe extending along the length of the nasal catheter 22 can be used to verify proper insertion of the nasal catheter in an x-ray or fluoroscopic image of the patient's airway.

Two oxygen connections 23 enable a flow of gas to be delivered through either end of the delivery tube 20. For example, the oxygen connections 23 can be female luer connectors as shown in the drawings. Removable cap plugs 24 are also provided at each end of the delivery tube to seal whichever end is not being used for delivery of gas. The entire nasopharyngeal catheter is held in place by two straps 25 that extend around the patient's head.

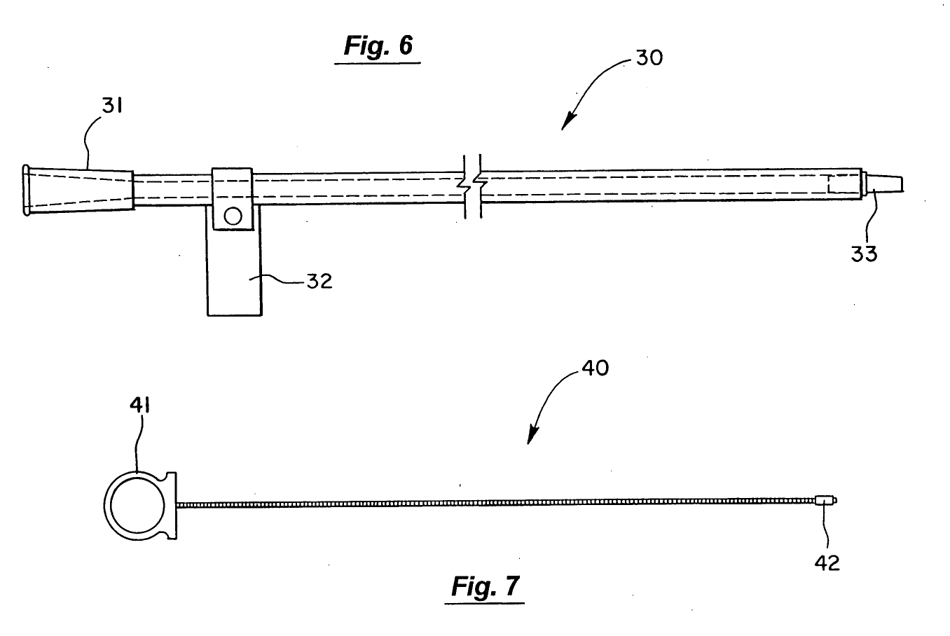

FIG. 6 is a side elevational view of the oxygen connecting tube 30 that can plugged into either of the oxygen connections 23 to supply a flow of gas through the delivery tube 20. In the preferred embodiment, the oxygen connecting tube 30 is made of soft PVC tubing and has a length of approximately 30 inches. The distal end of the oxygen connecting tube 30 has a male luer connector 33 for removably engaging the corresponding female luer connector of one of the oxygen connections 23. The proximal portion of the oxygen connecting tube 30 is equipped with a standard female luer connector 31 for connection to a conventional oxygen/air supply. A security clip 32 on the proximal portion of the oxygen connecting tube 30 can be secured to the patient's bed or clothing for safety.

Returning to FIG. 1, an air/oxygen supply 51 delivers gas at a flow rate of approximately 4 to 40 liters per minute. The flow passes through a heater 53 that maintains a desired temperature, and a humidifier 54 that maintains a desired relative humidity. The flow rate ultimately delivered through the oxygen connecting tube 30 to the nasopharyngeal catheter is determined by a flow regulator 55.

In the preferred embodiment, the air/oxygen supply 51 is liquid oxygen from a tank mixed with air from a compressor using a blender. The oxygen and air is mixed to approximately a 40 percent oxygen blend to maintain adequate blood oxygen although any mixture in a range of at least 21 to 100 percent oxygen could be so utilized. An oxygen analyzer monitors the oxygen content of the mixture exiting the blender. Should the oxygen content fall outside a desired range, the oxygen analyzer triggers a signal to alarm that notifies the patient or others of the incorrect oxygen content so that the content can be adjusted before harm occurs to the patient. The alarm can be local to the patient or suitably remote.

The blended oxygen/air mixture leaves the blender and goes into a flow regulator 55 that is adjusted to the desired flow rate for the patient, normally in a flow range from 4 to 40 liters per minute. A flow transducer is connected to the flow meter 55 to monitor the flow rate of the mixture exiting the flow meter. If the flow falls below or rises above the preselected flow, the flow transducer triggers a signal to an alarm remotely or locally so the flow can quickly be adjusted.

The air is directed from the flow meter through a flexible tube into a pop-off valve. The pop-off valve regulates the back pressure of the flow of the oxygen/air mixture in a preferred range of 2 to 25 psi. A pressure transducer is connected to the pop-off valve to monitor the back pressure of the mixture. If the pressure falls above or below the preselected range (i.e., the mixture is not flowing or if the pressure rises too high), the transducer triggers an alarm remotely or locally so the system can be properly adjusted.

The temperature of the mixture exiting the heater 53 is monitored by a temperature probe to maintain the mixture temperature at the desired value. The temperature probe is connected as close as practically possible to the nasopharyngeal catheter so the mixture can be monitored as near the patient as is feasible. Should the temperature fall below or rise above the selected range, the temperature probe triggers an alarm so the system can be adjusted. A humidity transducer monitors the humidity range of the mixture to trigger an alarm should the humidity of the mixture fall outside the selected range. The mixture then flows through the oxygen connecting tube 30 and into a nasopharyngeal catheter which has been inserted into the patient.

Each of the components are presently commercially available. The present invention is not meant to be limited by the identification of the particular components and other components can readily be used without departing from the scope of the invention.

The liquid oxygen tank(s) is readily obtainable from medical supply houses, such as the “LIBERATOR 53” liquid oxygen tank from Cryogenic Associates, New Prague, Minn. Liquid oxygen is preferable over high pressure oxygen cylinders due to the ease of handling and cost. The liquid oxygen is delivered by a flexible tubing into the blender, such as the “Bird 3800 Microblender,” manufactured by Bird Products Corporation, Palm Springs, Calif.

The oxygen is mixed in precise concentrations in the blender with air delivered through flexible tubing from a medical air compressor, such as the “6500 Air Compressor” also manufactured by Bird Products Corporation. Normally a concentration of 40 to 50 percent oxygen is desired although a range of at least 21 percent oxygen to 100 percent could be utilized. The blender has a control for setting the desired blend of oxygen to a predetermined value as determined by the physician or technician attending the patient. The setting will be such to maintain the proper blood oxygen level.

The transducers and alarms used to monitor the oxygen content, the flow rate, the pressure, the temperature and the humidity of the mixture are of types generally used in the medical field.

Attached to the blender is a flow regulator 55 which receives the blended oxygen/air mixture. The flow regulator 55 is adjustable to regulate the flow of the mixture, preferably from approximately 4 to 40 liters per minute. The mixture flows from the flow regulator 55 through flexible tubing into a pop-off valve assembly which regulates the back pressure of the mixture. The valve is adjustable to regulate the back pressure in a range of 2 to 25 psi. Should the pressure build up over 25 psi, the pop-off will bleed the excessive pressure of the mixture. The pop-off valve is preferably mounted directly to the chamber of the humidifier.

One such chamber is the “MR300” humidifying assembly (which can be disposable or non-disposable) by Fisher & Paykel, Auckland, New Zealand. Other conventional chambers could easily be used as well. The chamber is mounted on a humidifier heater base, such as the “MR620 Dual Servo Anesthesia Humidifier Heater Base” by Fisher & Paykel. This particular heater base is designed to limit the variation of the set temperature and humidity. An alternate heater/humidifier system is available from Vapotherm, Inc. of Annapolis, Md.

The mixture enters the humidifier from the pop-off valve and exits at a preferred humidity range of 80 to 100% with a preferred temperature range of 35 to 38 degrees Centigrade. This is approximately the body temperature of the patient. Maintaining the temperature and humidity at these ranges prevents the mixture from drying out the airway and lungs of the patient,

The components as described to this point are of a size and nature to be easily mounted on a wheeled cart. The related compact size of the system allows the system to be easily moved in either a home or hospital setting and is unobtrusive in the patient's home.

Alternatively, the liquid oxygen tanks can be replaced with an oxygen concentrator, such as is commercially available from Mountain Medical Equipment, Inc. of Littleton, Colo. The oxygen concentrator uses a molecular sieve material to separate oxygen from the remainder of air by the process of absorption. This eliminates the cost of replacing and refilling liquid oxygen tanks. In another embodiment, the liquid oxygen tanks and compressor can be replaced with an oxygen enricher. The enricher uses a permeable plastic membrane to separate oxygen and water vapor for the rest of the air by differences in gas diffusion rates. The units, such as the OECO high-humidity system manufactured by the Oxygen Enrichment Company, deliver a relatively constant 40 percent oxygen/air mixture directly to the flow regulator without the need for a blender.

As previously discussed, the flow of gas can be air or a mixture of air and oxygen. In some cases, pure oxygen may be detrimental in that it might tend to suppress spontaneous breathing by the patient. In another embodiment of the present invention, a mixture of oxygen and helium, or air and helium is supplied to the patient. As illustrated in FIG. 1, a helium supply 52 can be blended with gas from the air/oxygen supply 51. Helium is has a very low density that reduces the work of breathing. It is also chemically inert and very effective in penetrating into small spaces (e.g., alveoli) and past obstructions due to its density and viscosity.

FIG. 7 is a side elevational view of the cleaning rod 40 for cleaning the delivery tube 20 and nasal catheter 22. The cleaning rod 40 has a metal core with the wire-wound exterior, a ring-shaped handle 41 at its proximal end, and an atraumatic distal tip 42. The cleaning rod 40 can be inserted through either of the oxygen connections 23 to clean both branches of the delivery tube 20. The nasal catheter 22 can be cleaned by inserting the cleaning rod 40 through its distal tip.

The above disclosure sets forth a number of embodiments of the present invention. Other arrangements or embodiments, not precisely set forth, could be practiced under the teachings of the present invention and as set forth in the following claims.

1. (canceled)

2. The method of claim 23 wherein the catheter comprises a flexible plastic tube that can be cut to a desired length

3. The method of claim 2 wherein the catheter further comprises a plurality of markings indicating a series of common lengths for the catheter.

4. The method of claim 23 wherein the catheter further comprises a radio-opaque stripe.

5. The method of claim 23 wherein the delivery tube further comprises;
two opposing ends with connectors for removable attachment to a gas source; and
a cap removably insertable into a connector that is not attached to the gas source.

6. (canceled)

7. The method of claim 23 wherein the catheter further comprises a hydrophilic coating.

8. The method of claim 23 wherein the catheter has an inside diameter of approximately 3 mm.

9-10. (canceled)

11. The method of claim 23 wherein gas is supplied through the catheter at a back pressure of approximately 2 to 25 psi.

12-22. (canceled)

23. An open delivery method for providing a supplemental continuous flow of air/oxygen to a spontaneously breathing patient, the method comprising:
providing a delivery tube extending beneath the patient's nostril for delivering the flow of air/oxygen, said delivery tube having a connector for attachment to the catheter;
advancing a nasopharyngeal catheter through a patient's nostril until the distal tip of the catheter is visible through the patient's mouth below the patient's uvula;
cutting an end of the catheter to a desired length so that the distal tip of the catheter will have a desired position relative to the patient's uvula;
attaching the proximal end of the catheter to the connector on the delivery tube; and
supplying air/oxygen through the delivery tube and catheter at a flow rate of approximately 4 to 40 liters per minute into the patient's distal nasopharynx or oropharynx to supplement the patient's respiration without restricting the patient's spontaneous respiration through the patient's nasopharynx or oropharynx.

24-25. (canceled)

26. The method of claim 23 further comprising controlling the humidity of the air/oxygen supplied through the catheter.

27. The method of claim 23 further comprising regulating the temperature of the air/oxygen supplied through the catheter.

28. The method of claim 23 further comprising supplying helium through the catheter.
